(19) United States
(12) Patent Application Publication
(10) Pub. No.: US 2006/0149181 A1
(43) Pub. Date: Jul. 6, 2006

(54) Support spring

(76) Inventor: Norbert Guenther, Hergoldinger Strasse (DE)

Correspondence Address: BOYLE FREDRICKSON NEWHOLM STEIN & GRATZ, S.C., 250 E. WISCONSIN AVENUE, SUITE 1030, MILWAUKEE, WI, 53202, US

(21) Appl. No.: 10/544,855

(22) PCT Filed: Feb. 5, 2004
(86) PCT No.: PCT/DE04/00192

§ 371 Date: Aug. 5, 2005

(30) Foreign Application Priority Data
Feb. 7, 2003 (DE) 103-05-131.7

(51) Int. Cl. A61F 5/00

(52) U.S. Cl. 602/23; 602/27

(57) ABSTRACT

What is disclosed is a support spring comprising a calf-side end portion and a foot-side end portion interconnected through a heel part, in particular for articulatedly connecting a lower leg shell and a foot shell of an ankle-foot orthosis, wherein the spring rates of the support spring are different in accordance with the angle of rotation, as well as an ankle-foot orthosis for use of the support spring.

The invention relates to a support spring in accordance with the preamble of claim 1, and an ankle-foot orthosis in accordance with the preamble of claim 9.

The like support springs are employed, e.g., with ankle-foot orthoses for patients afflicted with deep paralysis, muscular ailments, infantile cerebral paresis, pathological disorders, neurological changes, or also with healthy persons, in order to aid the function of the plantar flexors. The ankle-foot orthosis supports the foot relative to the lower leg, and at the same time energy is absorbed by the support spring during the foot-down and stationary phases and output again during the toe-off phase.

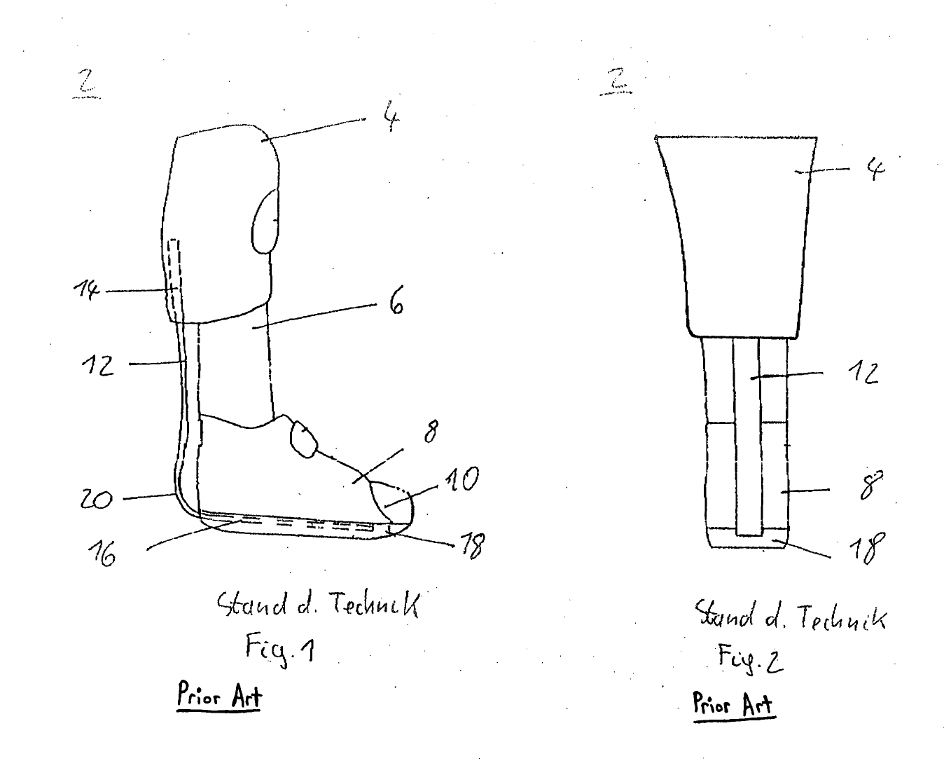

A-known support spring and an ankle-foot orthosis are shown in FIGS. 1 and 2. The figures originate from the applicant's ankle-foot orthosis SPRING and may be found in the catalog Medizinisches Verordnungsprogramm, Gottinger GmbH, 85604 Zorneding (DE). The ankle-foot orthosis 2 includes a lower leg shell 4 for encompassing a lower leg 6, and a foot shell 8 for retaining a foot 10. The two shells 4, 6 are articulatedly interconnected through a support spring 12 with a calf-side end portion 14 and a foot-side end portion 16, with the calf-side end portion 14 being received in the lower leg shell 4, and the foot-side end portion 16 in a sole 18 supporting the foot 10. In order to aid the spring effect, a heel part 20 of the support spring 12 is curved inversely.

It is a drawback in this known solution that the spring rate of the support spring is designed such as to result in a best possible support in the toe-off phase during walking. In accordance with the sequence of movements during walking, however, the support spring has to develop a higher supporting force in the toe-off phase than in the foot-down phase, so that with the known solution it is not possible to “gently” place the foot on the ground, and an irritating “push of the support spring into the knees”, or also a forceful recoil of the foot in the direction towards the knee takes place.

It is moreover a drawback that owing to the high spring rate, a patient wishing to execute an independent plantar flexion of the foot by muscular effort, such as in order to operate the pedals when driving an automotive vehicle, will necessarily always have to apply a high counterforce so as to oppose the spring force of the support spring.

It is the object of the present invention to furnish a support spring and an ankle-foot orthosis that eliminate the above mentioned drawback and that may be manufactured at low cost.

This object is attained through a support spring having the features in accordance with claim 1, and through an ankle-foot orthosis having the features in accordance with claim 9.

The support spring in accordance with the invention for articulatedly connecting a lower leg shell with a foot shell of an ankle-foot orthosis comprises a calf-side end portion and a foot-side end portion which are interconnected by a heel part. In accordance with the invention, the support spring possesses different spring rates in accordance with an angle of rotation of the foot about the talocrural joint, or ankle joint, towards the lower leg.

The essential advantage of the support spring in accordance with the invention resides in the fact that the support spring does not have a linear spring characteristic, so that the support spring develops a sufficient supporting force and a support in accordance with the respective walking or standing phase.

In a preferred manner, the support spring has a progressive spring characteristic, so that its spring rate increases with an increasing plantar flexion. Hereby it is possible to realize a “soft” setting down of the heel in the foot-down phase—small plantar flexion—and a “hard” propulsion of the foot in the toe-off phase—large plantar flexion.

In one preferred embodiment, the foot-side end portion is formed by two superposed branches. The branches are realized with the aid of a longitudinal slit extending from the free end of the end portion in a direction towards the heel part. In order to reduce notch effects in the heel part, the longitudinal slit preferably merges into a round expansion. Splitting the foot-side end portion results in a spring effect selectively only via one branch, or via both branches. The number of branches may be increased through additional longitudinal slits.

In a preferred manner, a ground-side branch is axially prolonged in comparison with a foot-side branch, and it may have a greater thickness when viewed in the vertical direction.

In order to support the spring effect of the support spring, the heel part is curved inversely relative to the calf-side and foot-side end portions, and is made of a fiber-reinforced, preferably carbon fiber-reinforced, plastic material.

An ankle-foot orthosis in accordance with the invention comprises a lower leg shell and a foot shell articulatedly interconnected through the intermediary of a support spring, in particular the support spring in accordance with the invention. In accordance with the invention, the ankle-foot orthosis has a stop which is consecutively contacted by portions of the support spring in the course of a plantar flexion, whereby different spring rates of the support spring may be realized.

In one preferred embodiment, a foot-side branch of the support spring contacts the stop, wherein the branch may only be brought into contact with the stop following a plantar flexion of about 10 degrees.

Further advantageous embodiments are subject matter of further appended claims.

In the following a more detailed explanation of the invention shall be given by referring to schematic representations, wherein:

FIG. 1 is a lateral view of a known ankle-foot orthosis including a known support spring,

FIG. 2 is a rear view of the ankle-foot orthosis of FIG. 1,

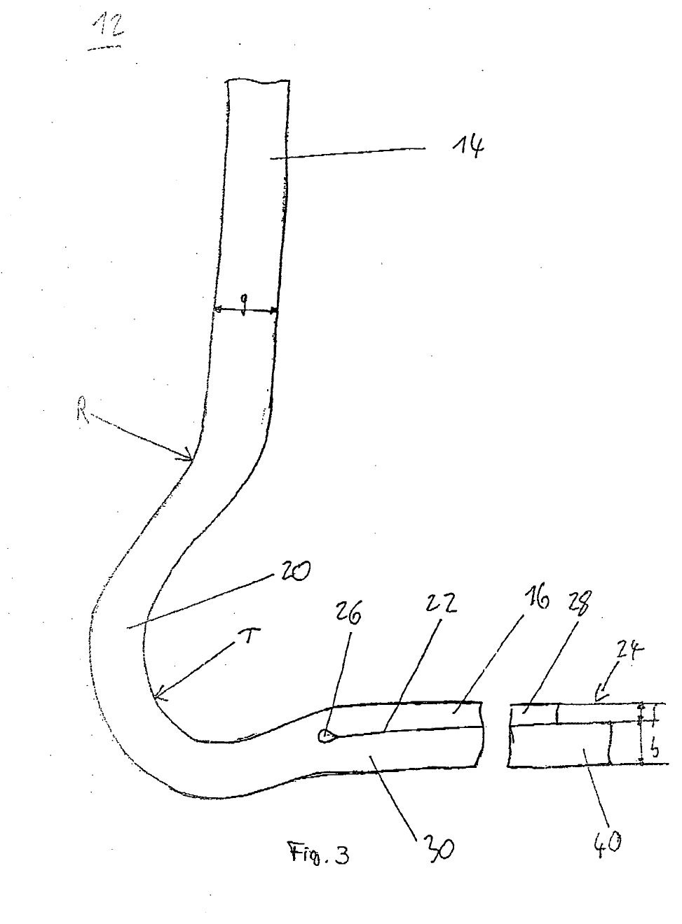

FIG. 3 is a lateral view of a support spring in accordance with the invention,

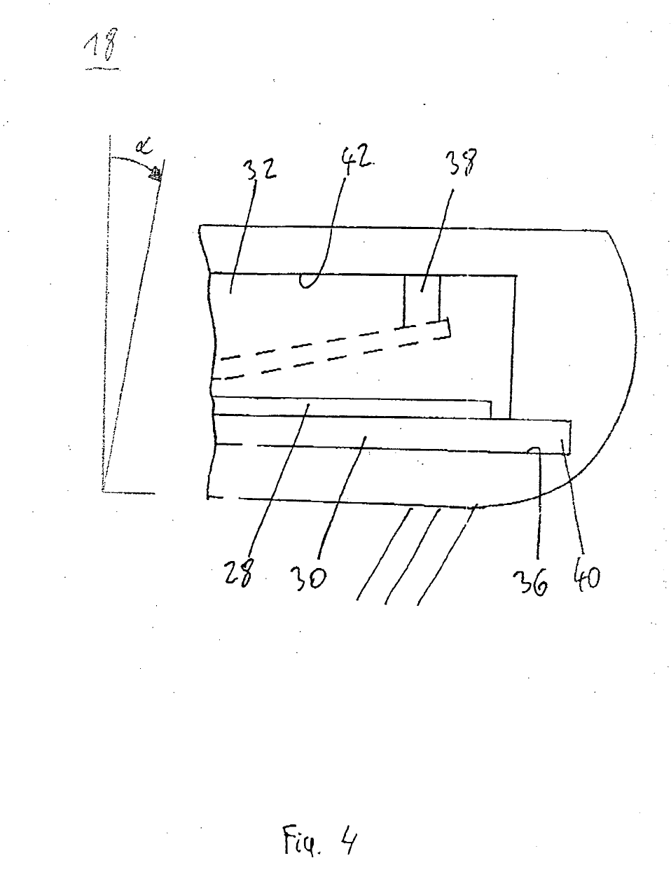

FIG. 4 is a cut-open, enlarged lateral view of a sole including a stop in accordance with the invention; and

FIGS. 5 through 8 show single gait phases of the human gait.

FIG. 3 shows a preferred embodiment of a support spring 12 in accordance with the invention. The support spring 12 roughly has an L-shaped structure with a calf-side end portion 14 and a foot-side end portion 16 in accordance with the invention, which are interconnected by an inversely curved heel part 20. The support spring 12 has the form of a leaf spring, with a fiber-reinforced plastic, e.g., carbon fiber-reinforced plastic, preferably being used as the material. This material is characterized by an excellent flexural strength at minimum weight and a high fatigue strength. In principle, however, it is also possible to use other suitable materials that will, however, as a rule have to be selected with a view to minimum weight and maximum fatigue strength.

The foot-side end portion 16 is in the longitudinal direction provided with a longitudinal slit 22 that extends from one free end 24 of the foot-side end portion 16 in a direction towards the heel part 20 and merges into a round expansion 26 so as to reduce notch effects. This accordingly results in the formation of two branches 28, 30 superposed in the vertical direction of the support spring 12, that each furnish a particular spring effect and may be tensioned singly or jointly depending on the degree of a plantar flexion, i.e., of a clockwise rotation of the foot about the talocrural joint, or an extension of the foot, respectively. The ground-side branch 30 is axially prolonged in comparison with the foot-side branch 28 by a prolongation 40.

In order to realize different spring forces of the two branches 28, 30, it is advantageous if the branches 28, 30 have different thicknesses f, b when viewed in the vertical direction. The overall thickness g of the support spring 12 is selected such as to be substantially constant over the entire length of the support spring 12, so that in each body portion 14, 16, 20 there applies: g=f+b.

The curvature of the heel part 20 is made up of two radii R and r. The radius r and the portion of the support spring 12 extending to the lower leg shell 4 (cf. FIGS. 1 and 2) essentially determine the mobility of the support spring 12 in the event of a plantar flexion, whereas the radius R primarily determines the mobility of the support spring 22 in the event of a dorsal extension, i.e., a counter-clockwise rotation of the foot about the talocrural joint. As a rule, the support spring 12 will be designed in such a way that a dorsal extension is substantially not supported while a plantar flexion is primarily supported. Mobility is thus placed in the range in the vicinity of the talocrural joint.

FIG. 4 shows a cut-open lateral view of a range near the toes of the sole 18 of an ankle-foot orthosis 2 in accordance with the invention, for use of the above described support spring 12 in accordance with FIG. 3. The lower leg shell 4 and the foot shell 8 do not substantially differ from the prior art in accordance with FIGS. 1 and 2, so that a renewed description thereof shall be omitted.

The sole 18 has a rectangular chamber 32 in which the foot-side end portion 16 of the support spring 12 is received by its branches 28, 30. For a secure connection of the support spring 12 with the sole 18, the ground-side branch 30 plunges with its prolongation into a corresponding recess 34 of the sole 18 that connects to the chamber 32. In a preferred manner, the prolongation 40 is secured in the recess 36 through positive engagement. The height of the chamber 32 is selected such that the foot-side branch 28 is at least in the rest position, i.e., when the support spring 12 is not tensioned, spaced apart from the opposite top surface 42 of the chamber 32. At the top surface 42, a stop 38 is arranged that extends in the direction towards the foot-side branch 28 and limits the angle, or angle of rotation, a of a plantar flexion from which the foot-side branch 28 is tensioned in addition to the ground-side branch 30 and thus develops a spring force that supersedes the one of the ground-side branch 30. In a preferred manner, the foot-side branch 28 contacts the stop 38 following a plantar flexion of α=10 degrees (indicated in dashed line).

It should be mentioned that a multiplicity of options are conceivable of how to realize the reception of the foot-side end portion 16 of the support spring 12 in the sole 18. The fundamental principle is, however, that the foot-side end portion 16 is fixedly connected with the sole 18 by at least one portion thereof—here: the ground-side branch 30—and is biased upon each plantar flexion of the sole 18, while at least one other portion of the foot-side end portion 16—here: the foot-side branch 28—is only biased following a defined plantar flexion, with the manifesting spring forces superseding each other to create an overall spring force of the support spring 12.

The function and operation of the support spring 12 of the invention in conjunction with the ankle-foot orthosis 2 of the invention is explained hereinbelow:

The ankle-foot orthosis 2 encompasses with its lower leg shell 4 and its foot shell 8 the lower leg 6 and the foot 10 of a patient. By means of the support spring 12 the shells 4, 8 are interconnected in an articulated manner, so that the patient can execute dorsal extensions and plantar flexions with his foot 10 about the talocrural joint, i.e., these movements are supported. The support spring 12 is fixedly received in the sole 18 by its ground-side branch 30, whereas the foot-side branch 28 only contacts the stop 38 following a plantar flexion of α≧10 degrees and thus develops a spring effect that supersedes with the spring effect of the ground-side branch 30. Owing to this split design of the foot-side end portion 16 and the individual tensioning of the branches 28, 30 in dependence on the angle of rotation a of the plantar flexion, the support spring 12 possesses a progressive spring characteristic, the spring rates of which increase with an increasing plantar flexion.

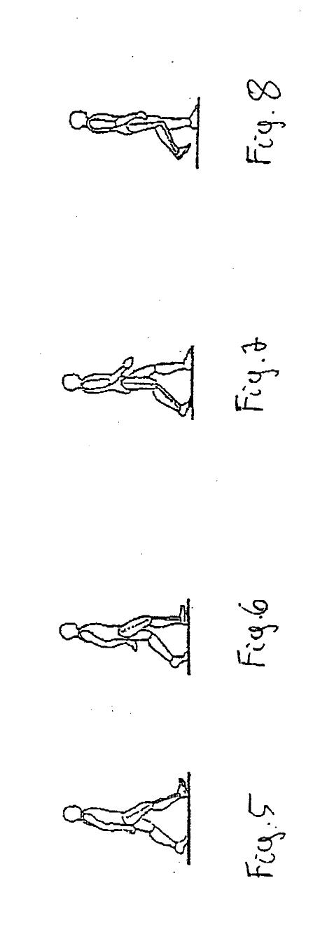

In the transposition of the function and effect in accordance with the invention to individual gait phases of human gait (FIGS. 5 to 8), this means that the support spring 12 is at the beginning of one gait cycle tensioned only through its ground-side branch 30 upon first contact of the heel with the ground (FIG. 5, right leg)—plantar flexion α<10 degrees. This is sufficient inasmuch as only part of the body weight needs to be absorbed. In the following loading reaction (FIG. 6), the patient's body weight is moreover absorbed, and absorption of the body weight increases with an increasing plantar flexion. Owing to the design of the support spring 12 in accordance with the invention, the foot-side branch 28 contacts the stop 38 following a plantar flexion of 10 degrees and thus equally develops a supporting force that supersedes the one of the ground-side branch 30. At the termination of the loading reaction, the entire sole of the foot touches the ground, and the body weight is shifted further to the stationary leg. The loading reaction is followed by the middle and end positions wherein the patient's center of gravity moves across the stationary leg, and the foot 10 performs a transition about the talocrural joint from the plantar flexion into a maximum dorsal extension of 10 degrees. In these phases the two branches 28, 30 contact each other, with both branches 28, 30 being engaged, so that a large dorsal extension that would be disadvantageous to the patient is prevented as a result of the spring force developed by the two branches 28, 30. In the transition to the forward swinging phase and in the forward swinging phase (FIG. 7) itself, the foot 10 again performs a transition from the dorsal extension into a plantar flexion, wherein the patient propels himself from the ground in the range of the ball of his foot and rolls off over the range of his toes. The plantar flexion usually amounts to up to 20 degrees. As a result of the split foot-side end portion 16 of the support spring 12 and of the stop 38 in the chamber 32, only the ground-side branch 30 is engaged up to a plantar flexion of 10 degrees, so that on the one hand it is made easier for the patients to initiate the plantar flexion on their own through muscular effort. On the other hand, the energy stored in the support spring 12 during the dorsal extension is preferentially output to only one branch—here: the ground-side branch 30—so that the energy absorbed during the dorsal extension supports the initiation of the plantar flexion with a corresponding degree of efficiency. In the subsequent initial (FIG. 8), middle, and final swinging phase, the foot 10 is raised from the ground, with the foot 10 performing a transition into a plantar flexion of 0 degrees as a result of the support spring 12 and being held there until the beginning of a new gait cycle.

A further example from which the positive effect of the progressive spring characteristic becomes clear, is the dorsal extension and plantar flexion of the foot in a seated activity, such as in operating the pedals for driving an automotive vehicle. Thanks to the support spring 12, the foot 10 with its toes is positioned at a distance from the ground while the lower leg 6 extends obliquely and the heel is planted down, so that the patient is well enabled to position his foot 10 above the pedals. Here the branches 28, 30 contact each other along the longitudinal slit 22, so that both branches 28, 30 are engaged for supporting this foot position, and accordingly the entire spring force of the support spring 12 is made available. Owing to the split foot-side end portion 16, however, the patient need not act against the entire spring force of the support spring 12 when depressing one of the pedals, but only against the spring force of the ground-side branch 30. Only starting from a plantar flexion of α≧10 degrees, the patient has to oppose a counterforce to the two spring forces of the branches 28, 30 and thus to the total spring force of the support spring 12.

By the combination of a suitable harmonization of the radii R and r of the curvature of the heel part 20 and of the lengths of the inversely curved portions with the split foot-side end portion 16 in accordance with the invention, the support spring 12 or the ankle-foot orthosis 2, respectively, may thus very simply be adapted to the individual requirements of patients.

What is disclosed is a support spring comprising a calf-side end portion and a foot-side end portion interconnected through a heel part, in particular for articulatedly connecting a lower leg shell and a foot shell of an ankle-foot orthosis, wherein the spring rates of the support spring are different in accordance with the angle of rotation, as well as an ankle-foot orthosis for use of the support spring.

1. A support spring comprising a calf-side end portion and a foot-side end portion which are interconnected by a heel part for an ankle-foot orthosis for articulatedly connecting a lower leg shell with a foot shell wherein the support spring has a spring rate that is different based on an angle of rotation.

2. The support spring according to claim 1, wherein the spring rate increases with an increasing angle of rotation, so that the support spring possesses a progressive spring characteristic.

3. The support spring according to claim 1, wherein a longitudinal slit in the foot-side end portion from a free end in a direction towards a heel part so as to result in two superposed branches.

4. The support spring according to claim 3, wherein the longitudinal slit merges into a round expansions.

5. The support spring according to claim 3, wherein the two branches have different axial lengths, with preferably the ground-side branch shaving a greater axial length.

6. The support spring according to claim, wherein the two branches have different thicknesses (f, b) when viewed in the vertical direction, with the ground side branch having a greater thickness than the foot-side branch.

7. The support spring according to claim 1, wherein the heel part is curved relative to foot-side and calf-side end portions.

8. The support spring according to claim 1, wherein the support spring is a leaf spring of fiber-reinforced plastic.

9. An ankle-foot orthosis comprising a lower leg shell and a foot shell articulatedly interconnected through the intermediary of at least one support spring (12), preferably a support spring (12) according to claim 1, wherein a stop which is consecutively contacted by portions of the support spring in the course of a plantar flexion, whereby different spring rates of the support spring are realized in accordance with an angle of rotation (α).

10. The ankle-foot orthosis according to claim 9, wherein the support spring contacts the stop by one branch of its foot-side end portion.

11. The ankle-foot orthosis according to claim 10, wherein the branch contacts the stop following a plantar flexion of about 10 degrees.

12. The support spring according to claim 8, wherein the leaf spring is a carbon-fiber reinforced plastic material.
